(19) United States
(12) Patent Application Publication
(10) Pub. No.: US 2006/0233875 A1
(43) Pub. Date: Oct. 19, 2006

(54) Taste masked sumatriptan tablets and processes for their preparation

(76) Inventors: Rajeev Shankar Mathur, Delhi (IN); T. Vijaya Kumar, Andhra Pradesh (IN); Sunilendu Bhushan Roy, Maharashtra (IN); Rajiv Malik, Wein (AT)

Correspondence Address: RANBAXY INC., 600 COLLEGE ROAD EAST, SUITE 2100, PRINCETON, NJ, 08540, US

(21) Appl. No.: 10/521,402

(22) PCT Filed: Jul. 17, 2003
(86) PCT No.: PCT/IB03/02838

§ 371 Date: Mar. 1, 2006

(30) Foreign Application Priority Data
Jul. 19, 2002 (IN) 759/DEL/2002

(51) Int. Cl. A61K 9/20; B27N 3/00

(52) U.S. Cl. 424/464; 264/109

(57) ABSTRACT

The technical field of the present invention relates to uncoated, taste masked sumatriptan tablets for oral administration and processes for their preparation. It also relates to wax polished sumatriptan tablets and processes for their preparation.

### FIELD OF THE INVENTION

The technical field of the present invention relates to uncoated, taste masked sumatriptan tablets for oral administration and processes for their preparation. It also relates to wax polished sumatriptan tablets and processes for their preparation.

### BACKGROUND OF THE INVENTION

Sumatriptan and its acid salts, particularly the succinate salt, are selective 5-hydroxytryptamine-1 (5HT1) agonists and are marketed as oral tablets in strengths of 25 and 50 mg equivalent to sumatriptan, under the trade name Imitrex®. It is indicated for the acute treatment of migraine attacks with or without aura in adults.

Sumatriptan and its physiologically acceptable salts have an unpleasant taste profile and, when administered orally, may intensify the nausea and vomiting associated with migraines. This limits the use of sumatriptan orally, which is considered to be the most widely accepted and convenient route of administration. Successful masking of the unpleasant taste is a key element for patients' acceptance and compliance of an oral dosage form. Prior art researchers have tried various techniques to mask the unpleasant taste of sumatriptan.

For example, PCT application WO 01/37816 discloses a process for the coating of sumatriptan tablet cores and tablets to provide taste masking of the sumatriptan. The process includes spraying a coating solution or suspension of a sugar, a starch, or a mixture of a sugar and a starch, onto tablet cores to obtain coated tablets. There is the proviso that film-forming agents in the suspension or solution are excluded. At page four of the '816 application, the inventors state that the solution or suspension of the coating mixture is sprayed onto the tablet cores in an amount sufficient to cover, e.g., uniform, the surface of the tablet cores. Similarly, U.S. Pat. No. 5,863,559 discloses a film coated solid dosage form of sumatriptan that the inventors state has the unpleasant taste substantially eliminated. The dosage form is disclosed as being a film coated tablet that includes a tablet core containing sumatriptan or a pharmaceutically acceptable salt or solvate thereof as active ingredient. The core is substantially covered with a coating that includes film forming polymers, such as hydroxypropyl methylcellulose, hydroxypropyl cellulose or methylcellulose, and copolymers of methacrylic acid and methyl methacrylate polymers.

In the prior art, a coating over the core tablet has been used to mask the bitter taste of sumatriptan. Though the coating may mask the unpleasant taste, if the thickness and composition of the coating is not properly controlled it may affect the disintegration and dissolution characteristics of the tablet. Further, the coating operation is a highly controlled and costly process. Preferably, film coatings should have good film properties and sufficient tensile strength to withstand the mechanical stresses associated with the processing, packing, transport and storage of the dosage forms. Moreover, the solution of the film-forming polymer must thoroughly wet the surface of the tablet's core and therefore must be finely atomized to spread well. Hence, only low concentrations of viscous film formers such as hydroxypropyl methylcellulose (HPMC) can be employed, and this results in longer processing times and high costs. Additionally, HPMC has other disadvantages, which include the wetting characteristics; adhesiveness to the tablet surface; pigment binding capacity; mechanical properties of the film; hygroscopicity; permeability to water vapor and oxygen; and a difference in disintegration times between film coated tablets and the core.

Similarly, sugar coating of tablet cores is a tedious process, is hygroscopic and requires greater weight build up to effectively taste mask. For conventional dosage forms, it is important that the disintegration of the tablet and the release of the active ingredient are not influenced by the coating itself.

### SUMMARY OF THE INVENTION

In one general aspect there is provided a process for preparing an uncoated sumatriptan tablet for oral administration. The process includes the steps of granulating sumatriptan or a physiologically acceptable salt with one or more diluents and/or binders to form granules; mixing the granules with one or more pharmaceutically acceptable excipients to form a mixture; and compressing the mixture to form a tablet.

Embodiments of the process of forming an uncoated sumatriptan tablet may include or more of the following features. For example, the process may ftuther include wax polishing of the tablet. The wax polishing may include spraying a solution or suspension of wax material onto the tablet and/or sprinkling a powder grade wax onto the tablet. The wax material may be one or more of shellac, modified shellac (opaglos), opaglos II, carnuba wax, bees wax, paraffin wax, and polyethylene glycol, and in particular may be modified shellac (opaglos). The total weight build up of wax polishing solid may be up to about 10% w/w, based on the total weight of the tablet.

Granulating may include dry mixing the one or more diluents and/or binders with sumatriptan and granulating with an aqueous and/or a non-aqueous solvent. The sumatriptan may be granulated with an aqueous and/or a non-aqueous solution or a suspension of one or more diluents and/or binders. The aqueous solvent may include water. The non-aqueous solvent may include one or both of alcohol and isopropyl alcohoL

The physiologically acceptable salt may be one or more of hydrochloride, hydrobromide, sulphate, nitrate, phosphate, formate, mesylate, citrate, benzoate, fumarate, maleate, tartrate and succinate salts, and may in particular be succinate (1:1).

The one or more diluents may be one or more of calcium carbonate, calcium phosphate-dibasic, calcium phosphate-tribasic, calcium sulfate, cellulose-microcrystaline, cellulose powdered, dextrates, dextrins, dextrose excipients, fructose, kaolin, lactitol lactose, mannitol, sorbitol, starch, starch pregelatinized, sucrose, sugar compressible, and sugar confectioners, and in particular may be lactose. The one or more binders may be one or more of methyl cellulose, hydroxypropyl cellulose, hydroxypropyl methyl cellulose, polyvinylpyrrolidone, gelatin, gum arabic, ethyl cellulose, polyvinyl alcohol, pullulan, pregelatinized starch, agar, tragacanth, sodium alginate, propylene glycol, and alginate, and in particular may be hydroxypropyl methylcellulose.

The one or more pharmaceutically acceptable excipients may be one or more of diluents, binders, disintegrants, lubricants, coloring agents, and flavoring agents. The disintegrant may be one or more of low substituted hydroxypropyl cellulose, carboxymethyl cellulose, calcium carboxymethyl cellulose, sodium carboxymethyl cellulose, croscarmellose sodium, starch, crystalline cellulose, hydroxypropyl starch, and partially pregelatinized starch, and in particular may be croscarmellose sodium. The lubricant may be one or more of stearic acid, magnesium stearate, calcium stearate, talc, hydrogenated caster oil, sucrose esters of fatty acid, microcrystalline wax, yellow beeswax, and white beeswax, and in particular may be one or both of talc and magnesium stearate.

The process may further include granulating and/or mixing a second active pharmaceutical ingredient with the sumatriptan.

In another general aspect there is provided a process for preparing one or more uncoated sumatriptan tablets for oral administration. The process includes the steps of spraying a solution or suspension of sumatriptan or a pharmaceutically acceptable salt in a solvent onto inert cores to form a first layer, blending the core having the first layer with one or more pharmaceutically acceptable excipients to form a blend, and compressing the blend to form an uncoated tablet.

Embodiments of the process may include one or more of the following features. For example, the solution or suspension of sumatriptan in a solvent may firther include one or more diluents and/or binders. The process may fiuther include creating a second layer on the cores having a first layer, the second layer including one or more diluents and/or binders.

The process may further include wax polishing the tablet, and polishing the tablet may include sprinkling a fine powder grade of a wax material on the tablet or spraying a solution or suspension of a wax material in organic solvent onto the tablet.

The inert core may include one or more of a sugar sphere, a non-pareil seed, celpheres, or a pharmaceutically acceptable inert insoluble, soluble or swellable material. The pharmaceutically acceptable inert core may include a non-pareil seed. The insoluble inert material may include one or more of sand, silicon dioxide, glass, microcrystalline cellulose, a plastic, and polystyrene. The soluble inert material may include one or more of a sugar, glucose, mannitol, lactose, xylitol, dextrose, and sucrose. The swellable inert material may be hydroxypropyl methylcellulose.

The process may further include spraying and/or blending a second active pharmaceutical ingredient with the sumatriptan.

In another general aspect there is provided a wax polished dosage form of sumatriptan. The dosage form includes sumatriptan or a physiologically acceptable salt; one or more pharmaceutically acceptable carriers or excipients; and a wax polish on the dosage form.

Embodiments of the dosage form may include one or more of the following features. For example, the wax polish may be a wax material. The wax material may be one or more of shellac, modified shellac (opaglos), opaglos II, carnuba wax, bees wax, paraffin wax, polyethylene glycol. The total weight buildup of wax material may be up to 10% w/w, based on the weight of tablet. The one or more pharmaceutically acceptable excipients or carriers may include one or more of diluent, binder, disintegrant, lubricant/glidant, coloring agent and flavoring agent. The wax polished dosage form of sumatriptan may further include a second active pharmaceutical ingredient in the dosage form.

In another general aspect, an uncoated, wax polished sumatriptan tablet includes a tablet core that includes about 10-200 mg of sumatriptan or a physiologically acceptable salt and one or more pharmaceutically acceptable carriers or excipients, and a wax polish on the tablet core. The wax polish includes an amount of from about 2 to 10% weight/weight of the tablet.

In another general aspect, there is provided an uncoated, taste-masked srrnatriptan tablet for oral administration that includes of an intragranular portion and an extragranular portion. The intragranular portion includes granules of sumatriptan or a pharmaceutically acceptable salt and one or more diluents and/or binders present in a sufficient amount to cause taste-masking of the sumatriptan or pharmaceutically acceptable salt. The extragranular portion includes one or more pharmaceutically acceptable excipients around the intragranular granules.

Embodiments of the uncoated, taste-masked sumatriptan tablet for oral administration may include one or more of the following features. For example, the one or more diluents and/or binders in the intragranular portion may completely encapsulate the sumatriptan or acceptable physiological salt or may substantially encapsulate the sumatriptan or acceptable physiological salt.

The intragranular portion and/or the extragranular portion may further include a second active pharmaceutical ingredient.

In another general aspect there is provided a method of treating or prophylactically treating a human suffering from a migraine condition. The method includes orally administering a wax polished dosage form of sumatriptan. The oral dosage form includes sumatriptan or a physiologically acceptable salt and a pharmaceutically acceptable carrier or excipient; one or more pharmaceutically acceptable carriers or excipients; and a wax polish on the dosage form.

In another general aspect there is provided a method of treating or prophylactically treating a human suffering from a migraine condition. The method includes orally administering an uncoated, taste-masked tablet of sumatriptan that includes an intragranular portion and an extragranular portion. The intragranular portion includes granules of sumatriptan or a pharmaceutically acceptable salt and one or more diluents and/or binders present in a sufficient amount to cause taste-masking of the sumatriptan or pharmaceutically acceptable salt. The extragranular portion includes one or more pharmaceutically acceptable excipients around the intragranular granules.

Embodiments of the method may include one or more of the following features. For example, the tablet may include about 10 mg to 200 mg of sumatriptan. The intragranular portion and/or the extragranular portion may firther include a second active pharmaceutical ingredient.

The details of one or more embodiments of the inventions are set forth in the description below. Other features, objects, and advantages of the invention will be apparent from the description and claims.

### DETAILED DESCRIPTION

Based on the above discussion of the prior art, the inventors recognized a need for a simpler and less expensive approach to masking the bitter taste of sumatriptan. Acting on this recognition, the inventors have now discovered a simple and economical process which effectively masks the unpleasant taste of sumatriptan without the need for any type of coating. Therefore, in one aspect there is provided a process for preparing an uncoated tablet for oral administration which effectively masks the taste of sumatriptan. In particular, the inventors have now discovered that taste masking properties can be imparted on the dosage form by granulating sumatriptan with one or more diluents and/or binders, mixing the granulated sumatriptan granules with other pharmaceutically acceptable excipients, and compressing to form a tablet. Alternatively, the granules can be filled into a capsule with pharmaceutically acceptable excipients to form a sumatriptan capsule.

The granulation may be carried out by dry mixing the one or more diluents and/or binders with sumatriptan and granulating with an aqueous and/or a non-aqueous solvent Alternatively, sumatriptan may be granulated with an aqueous and/or a non-aqueous solution/suspension of one or more diluents and/or binders. The aqueous solvent may be, for example, water, and the non-aqueous solvent may be, for example, alcohol or isopropyl alcohol.

The sumatriptan granules may be mixed with other pharmaceutically acceptable excipients and filled into a capsule or compressed to form a tablet. The tablet or capsule prepared above optionally may further be polished with a waxy material by sprinkling a fine powder grade of wax material, or spraying a solution/suspension of wax material in organic solvent, over the tablet or capsule. Polishing may be performed in airless spray equipment followed by air-drying in the spray equipment itself, or tray drying.

The processes described herein avoid a coating step and thereby reduce the processing time and costs associated with coating. Granulation with the one or more diluents and/or binders provides a uniform or a substantially uniform layer or encapsulation over or around the individual sumatriptan particles and thereby masks the unpleasant taste associated with sumatriptan. Nonetheless, optional wax polishing instead of a conventional coating may further provide additional taste masking, less tablet to tablet picking, good stability on storage, and aesthetic appeal to the tablet. It also reduces dust formation, which occurs during packaging of the tablets. Moreover, absence of any coating over the tablets helps to achieve the desired disintegration and dissolution characteristics without failure.

As used herein, the term “Sumatriptan” includes sumatriptan and its pharmaceutically acceptable salts. Such suitable salts include salts of inorganic or organic acids such as hydrochloride, hydrobromide, sulphate, nitrate, phosphate, formate, mesylate, citrate, benzoate, fumarate, maleate, tartrate and succinate salts. In particular, sumatriptan succinate salt (1:1) may be used.

In one embodiment, sumatriptan granules may be prepared by dry blending sumatriptan with one or more diluents and/or binders and granulating the blend with a solvent. The solvent can be an aqueous and/or a non-aqueous solvent.

In another embodiment, sumatriptan granules may be prepared by granulating sumatriptan with an aqueous/non-aqueous solution/suspension of one or more diluents and/or binders.

In yet another embodiment, sumatriptan granules may be prepared by dry blending sumatriptan with a portion of the one or more diluents and/or binders and granulating the blend with an aqueous/non-aqueous solution/suspension of the remaining portion of the one or more diluents and/or binders.

In yet another embodiment, sumatriptan granules may be prepared by spraying an aqueous/non-aqueous solution/suspension of one or more diluents and/or binders over the sumatriptan particles.

In yet another embodiment, sumatriptan granules may be prepared by spraying an aqueous/non-aqueous solution/suspension of sumatriptan alone or in combination with one or more diluents and/or binders onto inert cores.

In yet another embodiment, sumatriptan granules may be prepared by spraying an aqueous/non-aqueous solution/suspension of sumatriptan alone or in combination with one or more diluents and/or binders onto inert cores. An additional layer of one or more diluents and/or binders then can be deposited (e.g., spray) onto the inert cores that have been encapsulated or layered wit the first layer.

The sumatriptan granules prepared above may be filled into capsules of suitable size as such, made into a dispersion, or additionally may be blended with one or more pharmaceutically acceptable excipients and compressed into tablets or filled into capsules. The tablets or capsules optionally may be wax polished.

The optional wax polish includes a waxy material. Suitable waxy materials include one or more of shellac, modified shellac (opaglos), Opaglos II, carnuba wax, bees wax, paraffin wax, polyethylene glycol, and the like. One application is usually sufficient to obtain the desired effect. The preferred total weight build up of a wax polishing solid is up to about 10% w/w, based on the weight of the tablet.

Examples of suitable diluents useful for preparing sumatriptan granules include one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, starch pregelatinized, sucrose, sugar compressible, sugar confectioners and equivalents thereof.

Examples of suitable binders useful for preparing sumatriptan granules include one or more of methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, propylene glycol, alginate and equivalents thereof.

Examples of suitable disintegrants include one or more of hydroxypropyl cellulose, carboxymethylcellulose, calcium carboxymethylcellulose, sodium carboxyrnethylcellulose, croscarmellose sodium A-type (Ac-di-sol), starch, crystalline cellulose, hydroxypropyl starch, partly pregelatinized starch and equivalents thereof.

Examples of suitable lubricants/glidants include one or more of stearic acid, magnesium stearate, calcium stearate, talc, hydrogenated caster oil, sucrose esters of fatty acid, microcrystalline wax, yellow beeswax, white beeswax, silicon dioxide and equivalents thereof.

Examples of suitable coloring agents and flavoring agents include any FDA colors and flavors that are approved for oral use.

Examples of suitable inert cores include pharmaceutically acceptable inert cores available commercially or prepared from an inert material by processes of extrusion-spheronization, granulation and the like. Specific examples of commercially available inert cores include sugar spheres, non-pareil seeds, celpheres and the like. Alternatively, inert cores may be prepared from pharmaceutically acceptable inert soluble, insoluble and/or swellable materials, with or without pharmaceutically acceptable excipients. Examples of suitable soluble inert materials include sugars selected from glucose, mannitol, lactose, xylitol, dextrose, sucrose and equivalents thereof. Examples of suitable insoluble inert material include sand (silicon dioxide), glass, microcrystalline cellulose, plastic (polystyrene), and equivalents thereof. Examples of swellable inert material include hydroxypropyl methylcellulose and equivalents thereof. Inert cores may be of any geometric shape, although spherical cores are preferred for the ease of obtaining a uniform covering, layering or encapsulation.

Suitable solvents used for the preparation of sumatriptan granules may be selected from aqueous and/or non-aqueous solvents. Aqueous solvent used may be water whereas the non-aqueous solvent may be selected from one or more of ethanol, acetone, carbon tetrachloride, isopropyl alcohol, dichloromethane and equivalents thereof.

As described above and exemplified in more detail below, the process of forming taste masked sumatriptan dosage forms may be carried out by blending sumatriptan or its physiologically acceptable salts with approximately half of the quantity of one or more diluents and/or binders that are intended to be used; granulating the blend with the solvent; drying the granules and sifting to get the desired size; mixing the dried granules with rest of the one or more diluents and/or binders, and one or more disintegrants and/or lubricants; and compressing the blend to form tablets. Alternatively, sumatriptan may be granulated with the one or more diluents and/or binders dissolved or suspended in an appropriate solvent. These uncoated tablets optionally may be wax polished by spraying the wax solution or suspension using airless spray equipment. The wax polished tablets are either air dried or tray dried.

The following examples further exemplify the inventions and are not intended to limit the scope of the inventions.

### EXAMPLE 1

| INGREDIENTS | QUANTITY(mg/tablet) |
|---|---|
| INTRAGRANULAR | |
| Sumatriptan Succinate | 140.0 |
| eq. to 100 mg Sumatriptan | |
| Lactose Monohydrate | 133.0 |
| Purified Water | q.s. |
| EXTRAGRANULAR | |
| Microcrystalline Cellulose | 15.0 |
| Croscarmellose Sodium. | 3.0 |
| Magnesium Stearate | 3.0 |
| Talc | 6.0 |
| TOTAL WEIGHT | 300.0 |

Process for Forming the Tablets of Example 1:
1. Sumatriptan Succinate was sifted through a suitable mesh along with half of the above quantity of lactose and blended for 30 minutes to form a blend.
2. The blend was granulated using purified water to form granules.
3. The granules were dried at 60° C.
4. The dried granules were sized by passing them through a suitable mesh.
5. The sized granules were mixed with the remainder of the sifted lactose, the microcrystalline cellulose and the croscarmellose sodium for 20 minutes.
6. The granules formed in step 5 were mixed with magnesium stearate and talc for 5 minutes to form a blend.
7. The blend of step 6 was compressed using suitable tooling to form tablets.

The resulting tablets of Example 1 have an intragranular portion in which the sumatriptan is blended with the lactose monohydrate. The extragranular portion of the tablet partially or completely surrounds the granules.

### EXAMPLE 2

| INGREDIENTS | QUANTITY (mg/tablet) |
|---|---|
| INTRAGRANULAR | |
| Sumatriptan Succinate | 140.0 |
| Eq. to 100 mg Sumatriptan | |
| Lactose Monohydrate | 128.0 |
| Purified Water | q.s. |
| EXTRAGRANULAR | |
| Microcrystalline Cellulose | 32.5 |
| Croscarmellose Sodium | 6.0 |
| Magnesium Stearate | 4.5 |
| Talc | 3.0 |
| Colloidal Anhydrous Silica | 3.0 |
| TOTAL WEIGHT | 300.0 |

Process for Forming the Tablets of Example 2:
1. Lactose was dispersed in purified water.
2. Sumatriptan succinate was charged in a fluid bed processor and sprayed with the lactose dispersion by a top/bottomn/tangential spray to obtain granules.
3. The granules were mixed with the remaining excipients and compressed to form tablets.

The resulting tablets of Example 2 have an intragranular portion in which the sumatriptan succinate is substantially or completely encapsulated with the lactose monohydrate. The extragranular portion of the tablet partially or completely surrounds the granules.

### EXAMPLE 3

| INGREDIENTS | QUANTITY (mg/tablet) |
|---|---|
| INTRAGRANULAR | |
| Sumatriptan Succinate | 140.0 |
| eq. to 100 mg Sumatriptan | |
| Hydroxyproplyl Methylcellulose | 20.0 |
| Purified Water | q.s. |
| EXTRAGRANULAR | |
| Lactose Monohydrate | 108.0 |
| Microcrystalline Cellulose | 32.5 |
| Croscarmellose Sodium | 6.0 |
| Magnesium Stearate | 4.5 |
| Talc | 3.0 |
| Colloidal Anhydrous Silica | 3.0 |
| TOTAL WEIGHT | 300.0 |

Process for Forming the Tablets of Example 3:
1. Hydroxypropyl methylcellulose was dispersed in purified water.
2. Sumatriptan succinate was charged in a fluid bed processor and sprayed with the hydroxypropyl methylcellulose dispersion by a top/bottom/tangential spray to obtain granules.
3. The granules were mixed with the remaining excipients and compressed to form tablets.

The resulting tablets of Example 3 have an intragranular portion in which the sumatriptan succinate is substantially or completely encapsulated by the hydroxypropyl methyl cellulose. The extragranular portion of the tablet partially or completely surrounds the granules.

### EXAMPLE 4

| INGREDIENTS | QUANTITY (mg/tablet) |
|---|---|
| INTRAGRANULAR | |
| Nonpareil seeds | 100.0 |
| Sumatriptan Succinate | 140.0 |
| eq. to 100 mg Sumatriptan | |
| Lactose | 100.0 |
| Hydroxyproplyl Methylcellulose | 20.0 |
| Purified Water | q.s. |
| EXTRAGRANULAR | |
| Microcrystalline Cellulose | 17.5 |
| Croscarmellose Sodium | 12.0 |
| Magnesium Stearate | 4.5 |
| Talc | 3.0 |
| Colloidal Anhydrous Silica | 3.0 |
| TOTAL WEIGHT | 400.0 |

Process for Forming the Tablets of Example 4:
1. Sumatriptan and lactose were dispersed in purified water.
2. Nonpareil seeds were charged in a fluid bed processor and sprayed with the sumatriptan and lactose dispersion by a top/bottom/tangential spray.
3. Hydroxypropyl methylcellulose was dispersed in purified water.
4. The hydroxypropyl methylcellulose dispersion was sprayed by a top/bottom/tangential spray on the coated nonpareil seeds.
5. The coated nonpareil seeds were mixed with the remaining excipients and compressed to form tablets.

The resulting tablets of Example 4 have an intragranular portion in which the sumatriptan succinate and the lactose are sprayed on the nonpareil seeds, which are substantially or completely encapsulated with the hydroxypropyl methyl cellulose. The extragranular portion of the tablet partially or completely surrounds the granules.

The tablets prepared using the methods described in Examples 1-4 were optionally wax polished using any of the following techniques.
1. Carnuba Wax—0.5-2.0 mg/tablet
   Process: The sumatriptan tablets were charged in a polishing pan, warmed to 40-45° C. and sprinkled with fine powder grade of carnuba wax under rolling. The rolling was continued until uniform polishing was achieved.
2. Carnuba Wax—0.5-2.0 mg/tablet
   Carbon tetrachloride—qs
   Process: The carnuba wax was dissolved in a sufficient quantity of carbon tetrachloride and the mixture was applied on the sumatriptan tablets in a polishing pan under a hot stream of air (40-45° C.).
3. Polyethylene glycol—0.5-2.0 mg/tablet
   Isopropyl alcohol—qs
   Methylene chloride—qs
   Process: Polyethylene glycol was dissolved in a solution containing equal amounts of isopropyl alcohol and methylene chloride and the resulting mixture was applied on the sumatriptan tablets in a polishing pan under a hot stream of air (40-45° C).
4. Carnuba Wax—0.5-2.0 mg/tablet
   White wax—0.25-1.0 mg/tablet
   Carbon tetrachloride—qs
   Process: Carnuba wax and white wax (2:1 preferred ratio) were dissolved in a sufficient quantity of carbon tetrachloride and the resulting mixture was applied on the sumatriptan tablets in a polishing pan under a hot stream of air (40-45° C).
5. Opaglos
   Process: The sumatriptan tablets were charged in a polishing pan and polished using Opaglos, to a desired amount.

The dosage forms described herein can be used to treat a mammal, such as a human, suffering from or susceptible to conditions associated with cephalic pain such as cluster headache, chronic paroxysmal hemicrania, headache associated with vascular disorders, headaches associated with substances or their withdrawal (e.g., drug withdrawal), tension headache and, in particular, migraine headaches. The treatment involves oral administration of the pharmaceutical compositions described herein containing sumatriptan or a pharmaceutically acceptable salt or solvate thereof as the active ingredient. It should be understood that reference to treatment is intended to include prophylaxis as well as the treatment of expressed symptoms. The precise therapeutic dose of the active ingredient will depend on the age and condition of the patient and the nature of the condition to be treated. Moreover, the physician will have the discretion to vary the dose. In general, an effective dose for the treatment of conditions associated with cephalic pain, for example, acute treatment of migraine, is in the range of between 10 mg and 500 mg, particularly between 20 mg and 3 00 mg, and most particularly between 25 mg and 200 mg. For example, a suitable dose is a single or divided dose of 50 mg or 100 mg of the active ingredient per unit dose that is administered 1 to 4 times per day.

While several particular forms of the invention have been described, it will be apparent that various modifications and combinations of the invention detailed in the text can be made without departing from the spirit and scope of the invention. For example, though only conventional dosage forms of sumatriptan are disclosed, a person skilled in the art, with proper selection of modified release polymers may easily reap the advantages of the invention and hence would come within the scope of this invention. Modified release dosage forms may be prepared by using one or more modified release polymers. Examples of modified release polymers include cellulose derivatives such as ethyl cellulose, hydroxypropyl methylcellulose, hydroxypropylcellulose, methyl cellulose, carboxymethyl cellulose, hydroxymethylcellulose, hydroxyethylcellulose, hydroxypropyl methyl phthalate, cellulose acetate, cellulose triacetate, agar acetate, amylose acetate, cellulose acetate ethyl carbamate, cellulose acetate phthalate, cellulose acetate methyl carbamate, cellulose acetate succinate, cellulose acetate dimethylaminoacetate, cellulose acetate ethyl carbonate, cellulose acetate chloroacetate, cellulose acetate ethyl oxalate, cellulose acetate methyl sulphonate, cellulose acetate butyl sulphonate, cellulose acetate propionate, cellulose acetate diethylamino-acetate, cellulose acetate octate, cellulose acetate laurate, cellulose acetate p-toluenesulphonate, cellulose acetate butyrate; waxes such as polyethylene glycols; polymeric epoxides; copolymers of alkylene oxides and alkyl glycidyl ethers; polyglycols or polylactic acid derivatives; polysaccharides such as xanthan gum, guar gum, alginic acid; acrylic polymers such as eudragits and carbopols; and the like.

It may also be possible for a person skilled in the art to polish the dosage form using waxy materials other than those disclosed and they are thus expected to fall within the scope of this application. Moreover, the granules formed above can be filled into a capsule or made into another dosage form, such as a dispersion in a suitable medium.

A second active pharmaceutical ingredient can be used intragranularly, extragranularly, or both, provided the active pharmaceutical ingredients are chemically compatible to each other in the dosage form so prepared. For example, the sumatriptan of Example 1 can be granulated with a second active pharmaceutical ingredient. Alternatively, granules of the second active ingredient may be prepared separately and then the two types of granules may be blended together with extragranular excipients and compressed into tablets or filled into capsules.

The lactose of Example 2 can be mixed with a second active pharmaceutical ingredient and sprayed with the lactose onto the sumatriptan. Similarly, the hydroxypropyl methyl cellulose of Example 3 can be dispersed with a second active pharmaceutical ingredient and sprayed onto the sumatriptan. Moreover, based on the details of either Example 2 or 3, a blend of sumatriptan and a second active ingredient may be charged in a fluidized bed processor over which lactose/hydroxypropyl methylcellulose dispersion is sprayed. The second active ingredient also may be mixed with lactose in the dispersion and sprayed over sumatriptan. Further, as described above, the second active ingredient may be processed separately to prepare granules following the processes of Example 2 or 3 and finally the two different types of granules may be combined to prepare the dosage form.

The dispersion of sumatriptan and lactose of Example 4 and/or the dispersion of hydroxypropyl methyl cellulose of Example 4 can further include additional active pharmaceutical ingredients and be sprayed onto the seed and/or coated seed. Alternatively, the second active ingredient may be applied as a dispersion in a separate layer. Further, as in the examples above, the second active ingredient may be processed separately to prepare granules and finally the two different types of coated non pareils may be combined to prepare the dosage form.

An example of a suitable active pharmaceutical ingredient for administration with sumatriptan includes analgesics, such as ibuprofen, Tylenol®, and APAP (Acetaminophen). Moreover, other active pharmaceutical ingredients can be processed as described above to taste mask those ingredients. These active pharmaceutical ingredients include: antibiotics, such as penicillins, amoxicillin, and amoxicillin alone or in combination with clavulanic acid or clavulanic acid in the form of a potassium salt; penicillin V and therapeutically active derivatives, e.g., oxacillin, cloxacillin, flucoxacillin, dicloxacillin, and ampicillin; cephalosporins, e.g., cefaclor, cefixime, cephalexin, cephradine, cefadroxil, cefroxadine, cefdinir, cefpodoxime proxetil, and cefuroxime axetil; macrolides, e.g., erythromycin A, clarithromycin, azithromycin, and roxithromycin; antimigraines; and antipsychotics such as olanzapine. Further, it is contemplated that any single feature or any combination of optional features of the inventive variations described herein may be specifically excluded from the claimed invention and be so described as a negative limitation. Accordingly, it is not intended that the invention be limited, except as by the appended claims.

1. A process for preparing an uncoated sumatriptan tablet for oral administration, the process comprising the steps of:
granulating sumatriptan or a pharmaceutically acceptable salt with one or more diluents and/or binders to form granules;
mixing the granules with one or more pharmaceutically acceptable excipients to form a mixture; and
compressing the mixture to form a tablet.

2. The process according to claim 1, further comprising wax polishing the tablet.

3. The process according to claim 1, wherein granulating comprises dry mixing the one or more diluents and/or binders with sumatriptan and granulating with an aqueous and/or a non-aqueous solvent.

4. canceled

5. canceled

6. canceled

7. The process according to claim 1, wherein the pharmaceutically acceptable salt comprises one or more of hydrochloride, hydrobromide, sulphate, nitrate, phosphate, formate, mesylate, citrate, benzoate, fumarate, maleate, tartrate and succinate salts.

8. canceled

9. The process according to claim 1, wherein the one or more diluents comprises one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, starch pregelatinized, sucrose, sugar compressible, and sugar confectioners.

10. canceled

11. The process according to claim 1, wherein the binder comprises one or more of methyl cellulose, hydroxypropyl cellulose, hydroxypropyl methyl cellulose, polyvinylpyrrolidone, gelatin, gum arabic, ethyl cellulose, polyvinyl alcohol, pullulan, pregelatinized starch, agar, tragacanth, sodium alginate, propylene glycol, and alginate.

12. canceled

13. The process according to claim 1, wherein the pharmaceutically acceptable excipient comprises one or more of diluents, binders, disintegrants, lubricants, coloring agents, and flavoring agents.

14. canceled

15. canceled

16. canceled

17. canceled

18. canceled

19. canceled

20. The process according to claim 2, wherein wax material comprises one or more of shellac, modified shellac, opaglos II, carnuba wax, bees wax, paraffin wax, and polyethylene glycol.

21. canceled

22. The process according to claim 2, wherein the total weight build up of wax polishing solid comprises up to about 10% w/w, based on the total weight of the tablet.

23. The process according to claim 1, further comprising granulating and/or mixing a second active pharmaceutical ingredient with the sumatriptan.

24. A process for preparing uncoated sumatriptan tablets for oral administration, the process comprising the steps of:
spraying a solution or suspension of sumatriptan or a pharmaceutically acceptable salt in a solvent onto inert cores to form a first layer;
blending the core having the first layer with one or more pharmaceutically acceptable excipients to form a blend; and
compressing the blend to form a tablet.

25. The process of claim 24, wherein the solution or suspension of sumatriptan in a solvent further includes one or more diluents and/or binders.

26. The process of claim 24, further comprising creating a second layer on the cores having the first layer, the second layer comprising one or more diluents and/or binders.

27. The process of claim 25, further comprising creating a second layer on the cores having the first layer, the second layer comprising one or more diluents and/or binders.

28. The process of claim 24, further comprising polishing the tablet.

29. canceled

30. canceled

31. canceled

32. canceled

33. canceled

34. canceled

35. canceled

36. The process according to claim 24, further comprising spraying and/or blending a second active pharmaceutical ingredient with the sumatriptan.

37. A wax polished dosage form of sumatriptan, the dosage form comprising:
sumatriptan or a pharmaceutically acceptable salt;
one or more pharmaceutically acceptable carriers or excipients; and
a wax polish on the dosage form.

38. canceled

39. canceled

40. The wax polished dosage form of sumatriptan of claim 37, wherein the total weight buildup of wax material is up to 10% w/w, based on the weight of tablet.

41. canceled

42. canceled

43. canceled

44. The wax polished dosage form of sumatriptan of claim 37, further comprising a second active pharmaceutical ingredient in the dosage form.

45. An uncoated, wax polished sumatriptan tablet comprising:
a tablet core comprising about 10-200 mg of sumatriptan or a physiologically acceptable salt and one or more pharmaceutically acceptable carriers or excipients, and
a wax polish on the tablet core,
wherein the wax polish comprises an amount of from about 2 to 10% weight/weight of the tablet.

46. An uncoated, taste-masked sumatriptan tablet for oral administration, the uncoated tablet comprising:
an intragranular portion comprising granules of sumatriptan or a pharmaceutically acceptable salt and one or more diluents and/or binders present in a sufficient amount to cause taste-masking of the sumatriptan or pharmaceutically acceptable salt; and
an extragranular portion comprising one or more pharmaceutically acceptable excipients around the intragranular granules.

47. The uncoated, taste-masked sumatriptan tablet of claim 46, wherein the one or more diluents and/or binders in the intragranular portion completely encapsulate the sumatriptan or physiologically acceptable salt.

48. The uncoated, taste-masked sumatriptan tablet of claim 46, wherein the one or more diluents and/or binders in the intragranular portion substantially encapsulate the sumatriptan or physiologically acceptable salt.

49. The uncoated, taste-masked sumatriptan tablet of claim 46, wherein the intragranular portion and/or the extragranular portion further comprises a second active pharmaceutical ingredient.

50. A method of treating or prophylactically treating a human suffering from a migraine condition, the method comprising orally administering a wax polished dosage form of sumatriptan, the oral dosage form comprising:
sumatriptan or a physiologically acceptable salt and a pharmaceutically acceptable carrier or excipient;
one or more pharmaceutically acceptable carriers or excipients; and
a wax polish on the dosage form.

51. The method of treating of claim 50, wherein the tablet comprises about 10 to 200 mg of sumatriptan.

52. A method of treating or prophylactically treating a human suffering from a migraine condition, the method comprising orally administering an uncoated, taste-masked tablet of sumatriptan, the uncoated tablet comprising:
an intragranular portion comprising granules of sumatriptan or a pharmaceutically acceptable salt and one or more diluents and/or binders present in a sufficient amount to cause taste-masking of the sumatriptan or pharmaceutically acceptable salt; and
an extragranular portion comprising one or more pharmaceutically acceptable excipients around the intragranular granules.

53. The method of treating of claim 52, wherein the tablet comprises about 10 mg to 200 mg of sumatriptan.

54. The method of treating of claim 52, wherein the intragranular portion and/or the extragranular portion further comprises a second active pharmaceutical ingredient.
